(19) United States
(12) Patent Application Publication
(10) Pub. No.: US 2025/0290924 A1
(43) Pub. Date: Sep. 18, 2025

(54) MICROFLUIDIC DETECTION DEVICES INCLUDING SPIRAL FLOW PATHS

(71) Applicants: Research & Business Foundation Sungkyunkwan University, Suwon-si (KR); Ninebiowear Co., Ltd., Suwon-si (KR)

(72) Inventors: Nae-Eung LEE, Seoul (KR); Vinayagam Chinnamani MOTTOUR, Suwon-si (KR); Augustine SHINE, Suwon-si (KR)

(21) Appl. No.: 19/070,520

(22) Filed: Mar. 4, 2025

(30) Foreign Application Priority Data
Mar. 12, 2024 (KR) 10-2024-0034385

(51) Int. Cl. G01N 33/543; B01L 3/00

(52) U.S. Cl. CPC G01N 33/54386; B01L 3/502761; B01L 2200/0647; B01L 2200/16; B01L 2300/088; B01L 2300/0883; B01L 2400/0406

(57) ABSTRACT

Provided is a microfluidic detection device which has a spiral flow path and in which microfluid flows by a capillary phenomenon. The microfluidic detection device includes an upper layer configured to receive a fluid sample through a first inlet portion that protrudes on an upper side of a first substrate, the upper layer having a plurality of flow paths which is provided in a lower side of the first substrate and in which a washing solution moves. Furthermore, the microfluidic detection device includes a lower layer provided with a concave hole in an upper side of a second substrate such that the fluid sample is moved therethrough, the lower layer being configured to receive the fluid sample from a second inlet portion connected to the first inlet portion.

### BACKGROUND

### Technical Field

The present disclosure relates to a microfluidic detection device having a spiral flow path. More particularly, the present disclosure relates to a microfluidic detection device having a spiral flow path, the microfluidic detection device being configured such that a fluid sample and a detection antibody dAb are moved by a capillary phenomenon and are coupled to each other so that a first complex that is a complex of the detection antibody and a target molecule is formed, the microfluidic detection device being configured such that the first complex and a capture antibody cAb are coupled to each other so that a second complex that is a complex of a dAb-target-cAb is formed, and the microfluidic detection device being configured such that a waste solution that is not coupled as the second complex is removed by moving through the spiral flow path, thereby being capable of efficiently detecting a bio-marker.

### Description of the Related Art

A microfluidic detection device is used in various applications fields, and is particularly used as a key tool in life science and medical diagnosis in modern medical and life science research. The microfluidic detection device is mainly used for rapidly analyzing and diagnosing a fluid sample including a bio-marker.

Particularly, the microfluidic detection device has an advantage that the microfluidic detection device is capable of performing analysis with a small amount of a sample, and that the microfluidic fluid detection device is capable of quickly detecting a fluid sample with high sensitivity and specificity.

However, there is a problem that a conventional microfluidic detection device requires a manual processing of a bio-sample, an addition of a reagent, and a plurality of washing processes, so that there is a high probability that erroneous data is obtained due to an increase in complexity of the analysis process and an increase in worker interference.

In addition, in a passive microfluidic detection device, since the passive microfluidic detection device is vulnerable to a structure that eliminates bubble generation that hinders the flow of the fluid, the passive microfluidic detection device destabilizes the flow of the fluid, reduces the flow speed, and increases the pressure of the fluid, so that the performance of the passive microfluidic detection device is reduced and a situation where detection is impossible may occur.

In addition, since an antibody fixing function for fixing an antibody and the technology of removing a biomolecule that is not coupled to the antibody are required to be combined to each other for accurate data reading, there is a problem that a stable operation is difficult to be realized.

Accordingly, in order to stably output data from a fluid sample, a method for simplifying processing of the fluid sample to minimize worker operations and ensure easy device operation.

In addition, an integrated device capable of smoothly moving a fluid sample by a non-invasive method within a microfluidic detection device, fixing and coupling an antibody to the fluid sample for an immunoassay reaction, and efficiently detecting a bio-marker of the fluid sample by removing and washing an uncoupled biomolecule is required.

### SUMMARY

Accordingly, the present disclosure has been made keeping in mind the above problems occurring in the related art, and an objective of the present disclosure is to provide a microfluidic detection device having a spiral flow path, the microfluidic detection device being configured such that a fluid sample and a detection antibody are moved by a capillary phenomenon and are coupled to each other so that a first complex that is a complex of the detection antibody and a target molecule is formed, the microfluidic detection device being configured such that the first complex and a capture antibody are coupled to each other so that a second complex that is a complex of a dAb-target-cAb is formed, and the microfluidic detection device being configured such that a waste solution that is not coupled as the second complex is removed by moving through the spiral flow path, thereby being capable of efficiently detecting the target molecule.

The technical problem to be solved by the present disclosure is not limited to the above-mentioned problem, and other problems which are not mentioned will be clearly understood by those skilled in the art from the following description.

In order to achieve the objective of the present disclosure, according to an aspect of the present disclosure, there is provided a microfluidic detection device which has a spiral flow path and in which microfluid flows by a capillary phenomenon, the microfluidic detection device including: an upper layer configured to receive a fluid sample through a first inlet portion that protrudes on an upper side of a first substrate, the upper layer having a plurality of flow paths which is provided in a lower side of the first substrate and in which a washing solution moves; and a lower layer provided with a concave hole in an upper side of a second substrate such that the fluid sample is moved therethrough, the lower layer being configured to receive the fluid sample from a second inlet portion connected to the first inlet portion.

According to an aspect of the present disclosure, the upper layer may include: a pouch portion configured to supply the washing solution by a pressure; a collecting portion in which a waste solution containing the washing solution that is used is collected; a first spiral portion through which the waste solution passes; a valve portion configured to adjust a speed of the washing solution; a discarding portion in which the waste solution is stored; and a vent portion provided on a first side of the discarding portion and configured to discharge the pressure.

According to an aspect of the present disclosure, the pouch portion may include a washing fluid flow path in which the washing solution moves.

According to an aspect of the present disclosure, the valve portion may be provided in a shape symmetrically toward a center in a flow direction of the washing solution, and may be provided with a plurality of protrusions, thereby being capable of preventing backflow of the waste solution.

According to an aspect of the present disclosure, the vent portion may be provided as a hole that penetrates the upper side of the first substrate.

According to an aspect of the present disclosure, the lower layer may include: a loading portion in which a detection antibody is loaded so that the detection antibody is mixed in the fluid sample; a channel portion provided in a zigzag shape, the channel portion being configured to form a first complex by mixing the fluid sample and the detection antibody to each other; a second spiral portion that is a flow path provided such that the first complex and the washing solution are sequentially moved; and a coupling portion in which a capture antibody is loaded, the coupling portion having a predetermined depth such that a second complex is formed by coupling the first complex and the capture antibody to each other.

According to an aspect of the present disclosure, the microfluidic detection device may further include: a first flow path portion connecting the second inlet portion and the loading portion to each other; and a second flow path portion connecting the loading portion and the channel portion to each other.

According to an aspect of the present disclosure, the coupling portion may be configured such that the first complex and the capture antibody are filled in the coupling portion for a predetermined time and are coupled to each other.

According to an aspect of the present disclosure, the coupling portion may be provided at a lower side of the collecting portion so that the fluid sample containing the detection body not coupled as the second complex is collected in the collecting portion.

According to an aspect of the present disclosure, the waste solution may include: the fluid sample containing the detection antibody not coupled as the second complex; and the washing solution which is used and which contains impurities and bubbles.

According to an aspect of the present disclosure, the second spiral portion may be configured to move the first complex and the washing solution to the coupling portion.

According to an aspect of the present disclosure, the second spiral portion may connect the pouch portion and the coupling portion to each other, and may be configured such that a height of a flow path of the second spiral portion is reduced from the coupling portion to the pouch portion, thereby being capable of forming a structure preventing backflow of the first complex and the washing solution.

According to an aspect of the present disclosure, the second spiral portion and the first spiral portion may be provided such that a rotation direction of the second spiral portion and a rotation direction of the first spiral portion are opposite from each other.

According to an aspect of the present disclosure, the upper layer and the lower layer may be coupled to each other by chemical coupling at room temperature.

According to an aspect of the present disclosure, in the microfluidic detection device, the fluid sample and the detection antibody are moved by the capillary phenomenon and are coupled to each other so that the first complex is formed, the first complex and the capture antibody are coupled to each other so that the second complex that is a complex of the dAb-target-cAb is formed, and the waste solution that is not coupled as the second complex is removed by moving through the spiral flow path, so that the target molecule is capable of being detected efficiently.

The effects of the present disclosure are not limited thereto and it should be understood that the effects include all effects that can be inferred from the configuration of the present disclosure described in the following specification or claims.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a schematic view illustrating a microfluidic detection device including a spiral flow path according to an embodiment of the present disclosure;

FIG. 2 is a projection view illustrating an upper layer provided in the microfluidic detection device including the spiral flow path according to an embodiment of the present disclosure;

FIG. 3 is a perspective view illustrating the upper layer provided in the microfluidic detection device including the spiral flow path according to an embodiment of the present disclosure;

FIG. 4 is schematic views illustrating an inlet portion provided in the microfluidic detection device including the spiral flow path according to an embodiment of the present disclosure;

FIG. 5 is a schematic view illustrating a lower layer provided in the microfluidic detection device including the spiral flow path according to an embodiment of the present disclosure;

FIG. 6 is a perspective view illustrating the lower layer provided in the microfluidic detection device including the spiral flow path according to an embodiment of the present disclosure;

FIG. 7 is schematic views illustrating an intersecting portion provided in the microfluidic detection device including the spiral flow path according to an embodiment of the present disclosure;

FIG. 8 is schematic views illustrating a spiral portion provided in the microfluidic detection device including the spiral flow path according to an embodiment of the present disclosure; and

FIG. 9 is experiment results for proving effectiveness of the microfluidic detection device including the spiral flow path according to an embodiment of the present disclosure.

### DETAILED DESCRIPTION

Hereinafter, the present disclosure will be described in detail with reference to the accompanying drawings. However, the present disclosure may be implemented in many different forms and is not limited to the embodiments to be described herein. In addition, in order to clearly describe the present disclosure with reference to the drawings, parts irrelevant to the description are omitted, and similar reference numerals denote similar parts throughout the specification.

Throughout the specification, when a part is referred to as being “connected” (connect, contact, combine) to another part, it includes being “directly connected” to another part and “indirectly connected” to another part with still another part disposed therebetween. In addition, unless explicitly described to the contrary, the word “comprise” and variations such as “comprises” or “comprising” will be understood to imply the inclusion of stated elements but not the exclusion of any other elements.

The terminology used herein is for the purpose of describing particular embodiments only and is not intended to limit the present disclosure. As used herein, the singular forms are intended to include the plural forms as well, unless the context clearly indicates otherwise. In addition, it will be further understood that the terms “comprise”, “include”, “have”, and so on when used in the present application, specify the presence or absence of stated features, integers, steps, operations, elements, components, and/or combinations of them but do not preclude the possibility of the presence or addition of one or more other features, integers, steps, operations, elements, components, and/or combinations thereof.

Hereinafter embodiments of the present disclosure will be described in detail with reference to the accompanying drawings.

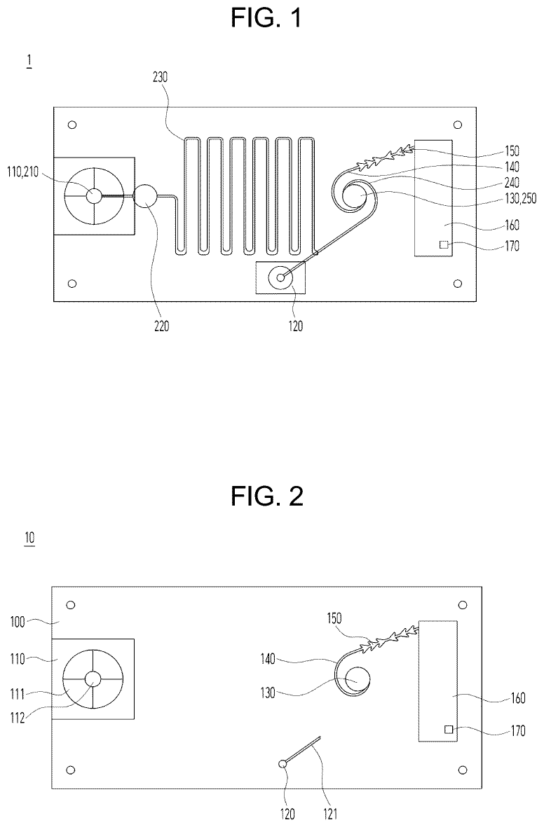

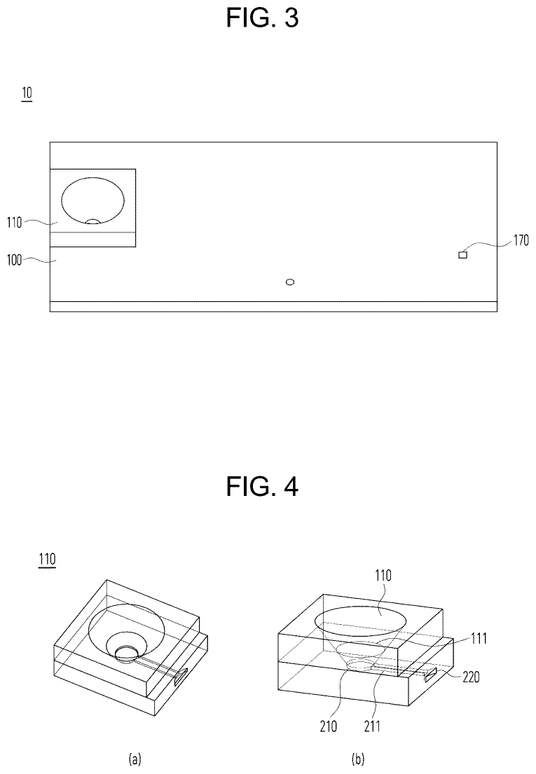

FIG. 1 is a schematic view illustrating a microfluidic detection device including a spiral flow path according to an embodiment of the present disclosure, FIG. 2 is a projection view illustrating an upper layer provided in the microfluidic detection device including the spiral flow path according to an embodiment of the present disclosure, FIG. 3 is a perspective view illustrating the upper layer provided in the microfluidic detection device including the spiral flow path according to an embodiment of the present disclosure, and FIG. 4A and FIG. 4B are schematic views illustrating an inlet portion provided in the microfluidic detection device including the spiral flow path according to an embodiment of the present disclosure.

Referring to FIG. 1 to FIG. 4B, a microfluidic detection device 1 may include an upper layer 10 and a lower layer 20.

In the microfluidic detection device 1, a flow path hole is provided between the upper layer 10 and the lower layer 20, so that a fluid sample may flow through the flow path hole. Furthermore, the upper layer 10 and the lower layer 20 may be bonded to each other by chemical coupling.

The upper layer 10 of the microfluidic detection device 1 may include a plurality of flow paths provided on a lower side of a first substrate 100, and the lower layer 20 may include a plurality of flow paths provided on an upper side of a second substrate 200. As the first substrate 100 and the second substrate 200 are bonded to each other, the plurality of flow paths of the first substrate 100 and the plurality of flow paths of the second substrate 200 are connected to each other, and a fluid sample introduced from a first inlet portion 110 provided on the first substrate 100 may pass through the flow paths of the second substrate 200.

Hereinafter, the microfluidic detection device 1 of the present disclosure will be described in more detail with reference to FIG. 2 and FIG. 3.

Referring to FIG. 2 and FIG. 3, the upper layer 10 may include the first substrate 100. Furthermore, the upper layer 10 may include the first inlet portion 110, a pouch portion 120, a collecting portion 130, a first spiral portion 140, a valve portion 150, a discarding portion 160, and a vent portion 170 that are provided on the first substrate 100.

The upper layer 10 may include the plurality of flow paths provided in the first substrate 100, and the first substrate 100 may be formed of polymethyl methacrylate (PMMA) that is a thermoplastic material. PMMA may be used as a substrate material of the microfluidic detection device 1 since PMAA has characteristics such as biological non-toxicity, biocompatibility, ease of surface modification, optical excellence, excellent hardness, ease of processing and mass production, and so on.

In addition, the first substrate 100 may be coated with APTES ((3-aminopropyl) triethoxysilane). The plurality of flow paths is provided in the first substrate 100, and the plurality of flow paths of the first substrate 100 may be provided by injection molding in which a PMMA material is injected at a high pressure into a heated mold cavity.

A fluid sample may be supplied from the first inlet portion 110 that protrudes on an upper side of the first substrate 100, and the first inlet portion 110 may include an inlet inner wall 111 and an inlet center portion 112.

The fluid sample may be introduced into the inlet inner wall 111 of the first inlet portion 110, may pass through the inlet center portion 112, and may be introduced into a plurality of flow path holes provided at the lower portion of the first substrate 100.

More particularly, the first inlet portion 110 which has a hexagonal shape having a predetermined height and which is coupled to the upper side of the first substrate 100 may be provided such that the fluid sample is capable of being introduced into the microfluidic detection device 1 by a force of gravity.

The first inlet portion 110 may have the inlet inner wall 111 having a conical shape in which a circular inlet thereof is gradually narrowed toward a lower portion thereof so that an inserted fluid sample flows downward, and the inlet inner wall 111 may be formed such that the inlet inner wall 111 is in contact with the inlet center portion 112.

A circumference of the inlet center portion 112 may be narrower than a circumference at a first side of the inlet inner wall 111, and the inlet center portion 112 may be provided as a hole having a circumference such that the inlet center portion 112 passes through the first substrate 100.

The fluid sample introduced into the first inlet portion 110 may pass through a second inlet portion 210 provided in the lower layer 20 by passing through the inlet center portion 112.

Meanwhile, the pouch portion 120 formed in a convex pouch shape may be provided on a first side of the first substrate 100.

The pouch part 120 is provided such that the pouch part 120 is filled with a washing solution, and may further include a washing solution flow path portion 121 through which the washing solution moves.

In addition, an inner portion of the pouch portion 120 may be formed in the convex pouch shape, and may be filled with the washing solution. The pouch portion 120 may be configured to supply the washing solution as a user pushes the pouch portion 120. The pouch portion 120 is attached to the upper portion of the first substrate 100 by using an adhesive tape, a hole is provided in the center of a lower portion of the pouch part 120, and the pouch portion 120 may be connected to the first substrate 100.

At this time, the washing solution flow path portion 121 is a flow path through which the washing solution supplied by the user passes, and a width of the washing solution flow path portion 121 may gradually increase as the washing solution flow path portion 121 extends away from the pouch portion 120. In addition, a phosphate buffered saline (PBS) solution with pH 7.0 may be used as the washing solution in the pouch part 120, but the washing solution is not limited thereto.

The washing solution supplied from the pouch portion 120 may be used in the plurality of flow paths provided in the second substrate 200, and may be collected at the collecting portion 130 provided on the first side of the first substrate 100.

As the washing solution is used in the collecting portion 130, a waste solution containing impurities and bubbles may be collected in the collecting portion 130. More particularly, as the washing solution passes through the second substrate 200, impurities and bubbles are collected into the first substrate 100, and the waste solution may be collected in the collecting portion 130.

The collecting portion 130 is connected to the first spiral portion 140, the first spiral portion 140 is provided as a flow path so that the waste solution is capable of passing through the flow path, and the first spiral portion 140 may be connected to the valve portion 150. Here, the waste solution may refer to a used washing solution which contains a fluid sample containing a detection antibody that is not coupled as a second complex and which contains impurities and bubbles. A fluid sample containing the detection antibody will be described later.

The first spiral portion 140 may be provided such that the first spiral portion 140 has a curved surface from a portion that is in contact with the collecting portion 130 and the width of the first spiral portion 140 gradually increases from the collecting portion 130 to the valve portion 150. Due to this structure of the first spiral portion 140, the waste solution may be moved without backflowing to the collecting portion 130 from the discarding portion 160 connected to the valve portion 150. At this time, the waste solution that passes through the first spiral portion 140 may be moved to the valve portion 150 by an external force applied to the pouch portion 120.

The first spiral portion 140 may be connected to the valve portion 150. The valve portion 150 is provided with a plurality of protrusions, and is coated with a hydrophobic material. Therefore, the valve portion 150 may stop the flow of a fluid after the fluid sample is filled in the collecting portion 130, and may prevent the backflow of the waste solution after washing.

A first side of the valve portion 150 is in contact with the first spiral portion 140, and a second side of the valve portion 150 is in contact with the discarding portion 160, so that the washing solution that passes through the valve portion 150 is capable of being moved from the first spiral portion 140 toward the discarding portion 160.

The valve portion 150 may be configured to adjust a speed of the washing solution. The valve portion 150 may have a shape symmetrical in a longitudinal direction and a transverse direction with respect to a direction through which the washing solution passes. More particularly, the transverse center is provided at the longitudinal center. Furthermore, a protrusion having an inner angle that forms an acute angle and having an outer angle that forms an obtuse angle is formed toward the longitudinal direction from the center in the longitudinal and transverse directions, and may obliquely protrude toward a peripheral direction from the center. The protrusion is formed such that a plurality of protrusions having “V” shapes is continuously arranged in the longitudinal direction, and the size of the “V” shape may be smaller as the distance from the center is increased, but is not limited thereto. In other words, the plurality of protrusions having the “V” shapes is arranged on the valve portion 150, a vertex of each of the plurality of protrusions is arranged in a direction toward the center of the valve portion 150. Furthermore, an “X” shape may be formed in the center of the valve portion 150 as the vertex of one of the plurality of protrusions and the vertex of one of the plurality of protrusions are in contact with each other. At this time, referring to FIG. 2 and FIG. 3, six protrusions having the “V” shapes may be provided, but the number of protrusions is not limited thereto.

In other words, the valve portion 150 is provided in a structure in which the width of the flow path of the valve portion 150 is gradually narrowed toward the discarding portion 160 from the collecting portion 130 and an angle of the “V” shape is gradually narrowed as the position of the protrusion is away from the center of the valve portion 150, thereby being capable of forming a structure that reduces a surface tension of the fluid sample. In addition, as the valve portion 150 is coated with the hydrophobic material, the flow of the fluid caused by the capillary phenomenon is stopped inside the valve portion 150. Therefore, after the collecting portion 130 is filled with the fluid sample, the flow of the fluid may be stopped in the valve portion 150. Furthermore, after a washing process is performed, the waste solution moved to the discarding portion 160 is prevented from backflowing to the collecting portion 130.

Such a structure may stop the movement toward the discarding portion 160 during collecting, and the total amount of fluid sample passing through the collecting portion 130 and remaining in the collecting portion 130, thereby being capable of providing an accurate quantification function.

Meanwhile, as described above, the vertex of the protrusion having the “V” shape and the other vertex of the protrusion having the “V” shape are in contact with each other so that the “X” shape is formed, and the shape of the protrusion may be formed such that the “V” shape is symmetrically formed with respect to the center point of the valve portion 150. In addition, the outer angle between the longitudinal direction of the valve portion 150 and the protrusion having the “V” shape may be 120 degrees, but is not limited thereto. Such a structure may be provided so that the fluid moving inside the valve portion 150 is capable of flowing in a constant direction.

Due to the shape of the valve portion 150, the flow of the fluid sample may be stopped within the valve portion 150 for a predetermined time. Furthermore, as the washing solution remains in the collecting portion 130, a reaction latency time required for forming a second complex C2 may be provided.

In addition, the waste solution that passes through the valve portion 150 may be stored in the discarding portion 160.

A rectangular concave space may be provided in the discarding portion 160 so that a used washing solution is stored therein, and an absorption pad may be embedded in the provided space so as to store the washing solution, so that backflow may be prevented.

The absorption pad provided inside the discarding portion 160 may be formed of synthetic resin such as non-woven fabric having pores therein for smooth absorption. As the absorption pad, at least one selected from a common non-woven fabric material such as polypropylene, polyethylene, polyester, acrylic-based synthetic resin, Teflon, polyvinyl chloride resin, polyethylene terephthalate, polyvinylidene fluoride, polytetrafluoroethylene, and a copolymer thereof, natural fiber, artificial fiber, synthetic fiber, glass fiber, and a combination thereof may be used, but is not limited to the words listed above.

The vent portion 170 is provided on a first side of the discarding portion 160, and a pressure generated while bubbles and the washing solution contained in the waste solution are supplied may be discharged from the vent portion 170. The vent portion 170 is provided as a hole that penetrates toward the upper side of the first substrate 100, and bubbles and pressure may be discharged to the outside of the vent portion 170.

More particularly, the pouch portion 120 is provided in the shape of the convex bag filled with the washing solution, and is configured such that the washing solution is supplied by applying a pressure as the pouch portion 120 is pressed. Furthermore, by the vent portion 170, a positive pressure generated by the flow of the washing fluid may be removed, and the bubbles contained in the waste solution may be discharged to the vent portion 170.

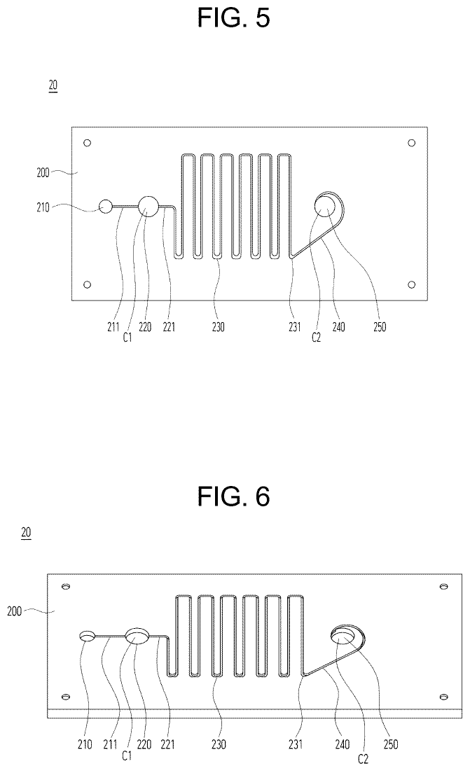

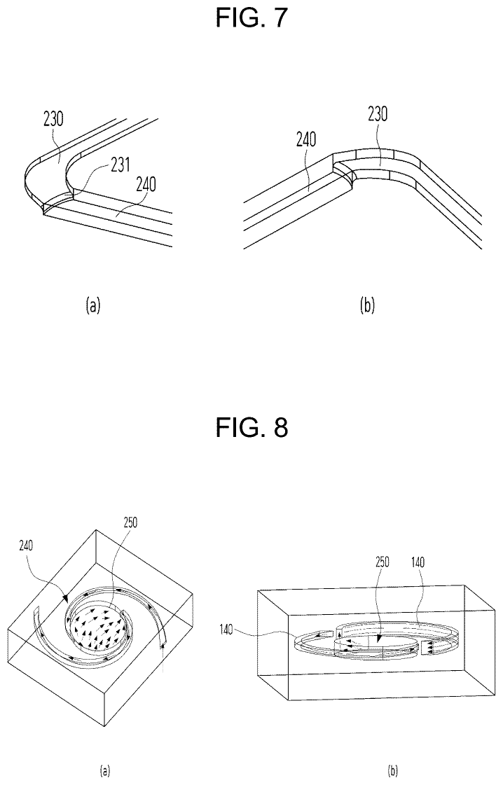

FIG. 5 is a schematic view illustrating a lower layer provided in the microfluidic detection device including the spiral flow path according to an embodiment of the present disclosure, FIG. 6 is a perspective view illustrating the lower layer provided in the microfluidic detection device including the spiral flow path according to an embodiment of the present disclosure, FIG. 7A and FIG. 7B are schematic views illustrating an intersecting portion provided in the microfluidic detection device including the spiral flow path according to an embodiment of the present disclosure, and FIG. 8A and FIG. 8B are schematic views illustrating a spiral portion provided in the microfluidic detection device including the spiral flow path according to an embodiment of the present disclosure.

Referring to FIG. 5 to FIG. 8B, the lower layer 20 may include the second substrate 200. Furthermore, the lower layer 20 may include the second inlet portion 210, a loading portion 220, a channel portion 230, a second spiral portion 240, and a coupling portion 250 that are provided on the second substrate 200.

The lower layer 20 may include the plurality of flow paths provided in the second substrate 200, and the second substrate 200 may be formed of styrene-ethylene-butylene-styrene (SEBS) that is a thermoplastic material. SEBS is an elastomer-based substrate material, and may be used as a substrate material of the microfluidic detection device 1 since SEBS has excellent flexibility, stretchability, and elasticity. Such a second substrate 200 may be coated with GPTMS ((3-glycidyloxypropyl) trimethoxysilane).

The second inlet portion 210 provided in the second substrate 200 has a shape corresponding to the shape of the first inlet portion 110, is connected to the first inlet portion 110, and may be configured such that the fluid sample is supplied to a plurality of flow path holes provided in the second substrate 200.

The second inlet portion 210 may be provided such that a circumference of the second inlet portion 210 corresponds to the circumference of the first inlet portion 110 so that the fluid sample introduced into the first inlet portion 110 is capable of being introduced into the second inlet portion 210. Particularly, the second inlet portion 210 may be formed such that the second inlet portion 210 is in contact with the inlet center portion 112.

At this time, the second inlet portion 210 may be in contact with a first flow path portion 211. The first flow path portion 211 may connect the second inlet portion 210 and the loading portion 220 to each other. The first flow path portion 211 may have a radius in which the fluid sample is capable of being moved by a capillary phenomenon, and the fluid sample moved through the first flow path portion 211 may be moved to the loading portion 220.

Glass fiber is loaded in the loading portion 220, and is capable of supplying a detection antibody. Glass fiber coupled to a detection antibody is seated on a floor surface of the loading portion 220, the detection antibody is a dAb (Detect-Antibody), and the detection antibody may be formed on the glass fiber by being coupled to sucrose.

For example, glass fiber (Fusion 5, Cytiva) is immersed in a solution containing 0.5% of BSA, 0.01% of Tween 20 surfactant, 0.01M of a PBS solution with pH 7.0, and a sucrose solution with a DI concentration of 20 wt. & at 37 degrees Celsius for two hours, and the detection antibody is loaded on the glass fiber and then is thermally treated at 37 degrees for an additional two hours, thereby being capable of providing the glass fiber. In this manner, the detection antibody with a volume of 5 μL and a concentration of 2.4 ug mL−1 may be loaded on the glass fiber.

In addition, the glass fiber may be provided with a diameter of 2 mm, and the loading portion 220 may be provided such that the loading portion 220 has a diameter equal to or larger than the diameter of the glass fiber so that the glass fiber is loaded on the loading portion 220.

In the loading portion 220 in which such a glass fiber is seated, as the fluid sample passes through the loading portion 220, the detection antibody contained in the glass fiber may melt and may be coupled to the fluid sample, and may be transferred to a second flow path portion 221.

A first side of the loading portion 220 is connected to the second flow path portion 221, and the fluid sample that passes through the loading portion 220 may be mixed with the detection antibody and may be moved to the channel portion 230.

The second flow path portion 221 may connect the loading portion 220 and the channel portion 230 to each other. The second flow path portion 221 may be provided at a predetermined depth within the second substrate 200 such that the fluid sample is capable of being moved by a capillary phenomenon, and the fluid sample moved through the second flow path portion 221 may be moved to the channel portion 230.

When particles exist on a fluid interface, an interface transformation around the particles occurs and a surface free energy increases, and the capillary phenomenon may refer to a force that is a driving force in a phenomenon in which the particles are arranged and moved in a direction in which the total surface area of the fluid interface is reduced except for an area occupied by the particles. By such a driving force, the fluid sample may move in the plurality of flow paths without an external power supply.

The channel portion 230 is provided in a zigzag shape, and a first complex C1 may be formed by mixing the fluid sample with a detection antibody in the channel portion 230. At this time, the first complex C1 may be formed as a complex formed by coupling a target molecule of the fluid sample with a detection antibody.

The channel portion 230 is formed such that the channel portion 230 has a width the same as the width of the second flow path portion 221, and may be formed in the upper side of the second substrate 200 by connecting a plurality of longitudinal flow paths and a plurality of transverse flow paths to each other so as to maintain a latency time for forming the first complex C1.

At this time, the transverse flow path may be formed in a direction the same as the entrance direction of the second flow path portion 221, may be formed with a narrower spacing than that of the longitudinal flow path, and a splicing portion having a curved shape may be formed between the longitudinal flow path and the transverse flow path.

As the channel portion 230 is formed as the narrow passage, the mixing efficiency of the fluid sample and the detection antibody may be increased while the fluid sample and the detection antibody introduced into the channel portion 230 pass through the channel portion 230.

Meanwhile, the first flow path portion 211, the second flow path portion 221, and the channel portion 230 provided in the second substrate 200 are provided with hydrophilic surfaces, and the flow path having the hydrophilic surface allows the fluid sample to smoothly flow, so that an environment in which the fluid sample is capable of being moved to the collecting portion 130 may be provided.

In addition, the first complex C1 passing through the channel portion 230 may pass through the second spiral portion 240, and may be transferred to the coupling portion 250. [The first complex contained in the fluid sample is coupled to a capture antibody fixed to the substrate and, at this time, the first complex is transferred by a diffusion phenomenon.]

At this time, an intersecting portion 231 may be formed at a point where the channel portion 230 and the second spiral portion 240 are in contact with each other.

The intersecting portion 231 is a point where the pouch portion 120 formed on the first substrate 100, the channel portion 230 of the second substrate 200, and the second spiral portion 240 of the second substrate 200 are in contact with each other, and may form a section to which the first substrate 100 and the second substrate 200 are connected.

Such an intersecting portion 231 may receive the washing solution from the pouch portion 120 of the first substrate 100. Starting from the intersecting portion 231, the first complex C1 supplied from the channel portion 230 may be supplied preferentially in the second spiral portion 240, and the washing solution supplied from the pouch portion 120 may be sequentially supplied.

The second spiral portion 240 may be provided as a spiral flow path.

At this time, a predetermined section of the second spiral portion 240 is formed as a straight line. Furthermore, as the position of the second spiral portion 240 is positioned close to the coupling portion 250, the width of the flow path of the second spiral portion 240 is gradually narrowed and the second spiral portion forms a curved surface having a spiral shape, and may be in contact with the coupling portion 250. Furthermore, a point of the second spiral portion 240 in contact with the coupling portion 250 may be provided as an end of a semi-circle beginning from a point where the curved surface is formed. In addition, in the shape in which the width of the second spiral portion 240 is gradually narrowed, the flow speed of the washing solution is increased as the washing solution is positioned close to the coupling portion 250, so that the cleaning efficiency may be increased.

At this time, the second spiral portion 240 may have a structure in which a height of the second spiral portion 240 is gradually reduced from the intersecting portion 231 provided in the first substrate 100 toward the coupling portion 250 provided in the second substrate 200 and in which a height of the flow path of the second spiral portion 240 is gradually reduced from the coupling portion 250 toward the pouch portion 120. In the second spiral portion 240, the washing solution is supplied by an external force applied to the pouch portion 120 that is connected to the intersecting portion 231, and a backflow of the washing solution occurs due to a negative pressure that removes an external force. Such a height structure of the second spiral portion 240 has an effect of increasing the backflow resistance. Such a second spiral portion 240 may be provided with the hydrophilic surface, so that liquid passing through the second spiral portion 240 may flow smoothly. As the speed of the washing solution passing through the second spiral portion 240 increases, the cleaning efficiency may be increased while a vortex is formed in the coupling portion 250 due to generation of a ram pressure in a vertical direction.

Meanwhile, the first complex C1 preferentially passes through the second spiral portion 240, and the washing solution may pass through second spiral portion 240 after a predetermined time. That is, after the first complex C1 remains in the collecting portion 130 for a predetermined time, the washing solution may be sequentially supplied with a time interval for performing the washing process. Here, the washing process may be a process in which the washing solution is sprayed into the collecting portion 130 so as to remove bubbles and the detection antibody that is not coupled to the second complex.

In other words, the shape of the second spiral portion 240 increases the reaction area and is capable of improving the mixing efficiency as the movement path of the first complex C1 and the washing solution is longer than a straight shape. In addition, since the second spiral portion 240 has the curved shape, turbulence may be prevented from occurring in the flow of the liquid, and the flow speed of the washing solution passing through the second spiral portion 240 may be increased, so that the cleaning efficiency of the washing solution may be increased.

Such a second spiral portion 240 may move the first complex C1 to the coupling portion 250 while preventing the generation of bubbles and preventing the generation of backflow.

The coupling portion 250 may be provided such that the capture antibody is loaded in the coupling portion 250 so that the first complex C1 and the capture antibody are coupled to each other. The coupling portion 250 may be provided at a lower side of the collecting portion 130 such that impurities of the first complex C1 and the capture antibody are collected in the collecting portion 130.

The coupling portion 250 may be provided as a cylindrical space, and a substrate coupled to the capture antibody may be attached to a floor portion of the coupling portion 250. The substrate coupled to the capture antibody cAb (Capture-Antibody) is attached to the floor portion of the coupling portion 250, and the substrate may be formed of an Au/Ag ND substrate.

For example, the Au/Ag ND substrate is immersed in 10 mM of an ethanol solution of 11-MUA (11-mercaptoundecanoic acid) for at least 12 hours to create a self-assembled monolayer (SAM) of a uniform amine group, the SAM layer is immersed in a solution formed by mixing 50 mM of EDC and 200 mM of NHS at a 1:1 volume ratio, the SAM layer is washed with deionized water DI and is dried with N₂, and then the SAM layer is fixed with 6 g/mL concentration of a cAb solution (anti-AIMP-2) and is stored at 4 degrees Celsius for 12 hours, thereby forming the substrate to which the capture antibody is coupled. In a substrate coupled to the capture antibody and provided in this manner, the antibody may be efficiently fixed by using protein G/11-MUA SAM.

At this time, the capture antibody is fixed to the substrate of the coupling portion 250, and the uncoupled detection antibody may be removed during the washing process.

In the coupling portion 250, the first complex C1 and the capture antibody are filled for a predetermined time and may be coupled to each other, and may form the second complex C2. At this time, the predetermined time may preferably be a latency time of 20 minutes to 30 minutes. Such a latency time may be provided such that the first complex C1 and the capture antibody form the second complex C2 by being coupled to each other by a diffusion phenomenon.

To this end, the coupling portion 250 is provided as a cylindrical shape having a predetermined depth, and the second complex C2 may be formed on the substrate provided at the lower side of the coupling portion 250.

At this time, the second complex C2 may be formed as a complex of a dAb-target-cAb, and the fluid sample containing the detection antibody that is not coupled as the second complex C2 may be collected in the collecting portion 130. The coupling portion 250 and the collecting portion 130 may be provided as an integral structure with a cylindrical shape on a cutting surface of the microfluidic detection device 1.

More particularly, the fluid sample containing the detection antibody that is not coupled as the second complex C2 in the coupling portion 250 may be collected to the collecting portion 130, the washing solution may be supplied while the pouch portion 120 is operated, and the fluid sample containing the detection antibody that is not coupled as the second complex C2 may be collected to the discarding portion 160 through the first spiral portion 140 by an external force applied to the pouch portion 120. Meanwhile, the first spiral portion 140 has a hydrophilic surface similar to the second spiral portion 240, so that the washing solution and the fluid sample that contains the detection body not coupled as the second complex C2 are capable of flowing by the pressure applied to the pouch portion 120.

In addition, the first spiral portion 140 and the second spiral portion 240 are provided such that a rotation direction of the first spiral portion 140 and a rotation direction of the second spiral portion 240 are opposite from each other. Therefore, a structure in which the first complex C1 is introduced into the coupling portion 250 and the waste solution is discharged to the collecting portion 130 is provided, so that a structure that is easy to wash may be provided.

Meanwhile, the upper layer 10 and the lower layer 20 may be coupled to each other by chemical coupling at room temperature. For example, the first substrate 100 and the second substrate 200 are precisely aligned and are pressed with the pressure of 150 kPa for two hours, so that an organic silane group layer is formed between the APTES layer of the first substrate 100 and the GPTMS layer of the second substrate 200, thereby being capable of attaching the first substrate 100 and the second substrate 200 to each other at room temperature.

In addition, as a bonding method for bonding the first substrate 100 and the second substrate 200 to each other, a bonding method using an organic solvent and a local laser bonding method may be used. Such a bonding method may prevent a problem in which an antibody is decomposed or damaged at a high temperature and which occurs in a bonding method such as a thermal attachment method used conventionally, an attachment method using a solvent, an attachment method ultrasonic laser, and so on.

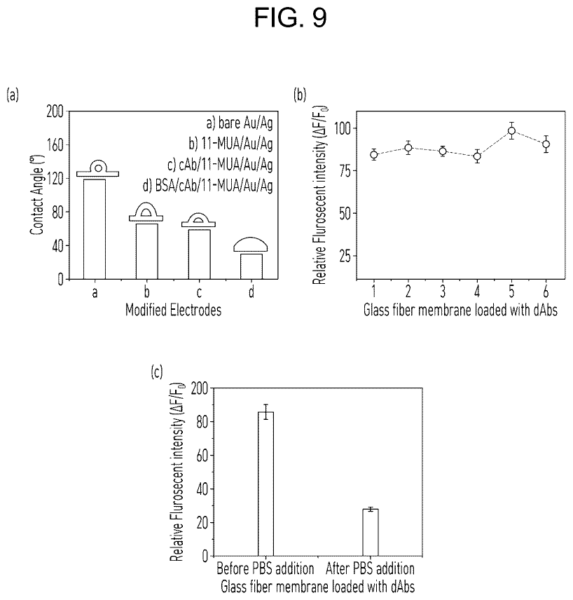

FIG. 9A to FIG. 9C are experiment results for proving effectiveness of the microfluidic detection device including the spiral flow path according to an embodiment of the present disclosure.

FIG. 9A is a result of measuring a contact angle (CA) with an atomic force microscope (AFM) by using a sessile drop method in order to check a fixing effect of a capture antibody in a complex portion. While a contact angle (CA) of an Au/Ag ND substrate is about 119 degrees, a contact angle (CA) of an 11-MUA SAM-treated substrate is about 66 degrees, so that it is confirmed that the substrate has hydrophilic properties after the SAM surface treatment is performed.

FIG. 9B is a result of measuring a coefficient of variation of a fluorescence signal measured in a loading portion on which a detection antibody is loaded. When the measurement is performed within the sucrose concentration range of 5 wt. % to 40 wt. %, the coefficient of variation of the fluorescence signal was less than 5%, so that it is derived that the sucrose concentration of 20 wt. % is the optimal condition for fixing the detection antibody on glass fiber.

In addition, FIG. 9C is a graph measuring a relative fluorescence change after the washing solution is flushed to the loading portion. When the washing solution flows, the detection antibody is released from the glass fiber, so that the fluorescence intensity is reduced by half.

In the microfluidic detection device 1 according to an embodiment of the present disclosure, the fluid sample and the detection antibody are moved by a capillary phenomenon and are coupled to each other so that the first complex is formed, the first complex and the capture antibody are coupled to each other so that the second complex that is a complex of the dAb-target-cAb is formed, and the waste solution that is not coupled as the second complex is removed by moving through the spiral flow path, so that the microfluidic detection device capable of efficiently detecting the target molecule may be provided. The microfluidic detection device 1 images fluorescence by using a digital fluorescence microscope on the dAb-target-cAb complex and quantifies the target concentration of the immunoassay reaction by measuring fluorescence intensity, and the detected target molecule may be fluorescently marked on the substrate provided in the coupling portion 250.

In addition, since the target molecule in which impurities are removed is detected in the microfluidic detection device 1, a fluorescence signal of impurities is reduced, and only a fluorescence signal of the target molecule is detected, so that there is an effect that the accuracy in detecting the fluorescence intensity is increased.

The above descriptions on the present disclosure are for illustration, and those skilled in the art to which the present disclosure pertains may understand that the descriptions may be easily modified into other specific forms without changing the technical idea or essential features of the present disclosure. Therefore, it should be understood that the embodiments described above are illustrative in all respects and not restrictive. For example, each component described as a single type may be implemented in a separated form, and likewise components described as distributed may be implemented in a combined form.

The scope of the present disclosure is indicated by the following claims rather than the above detailed description, and all changes or modifications derived from the meaning and scope of the claims and their equivalents should be interpreted as being included in the scope of the present disclosure.

What is claimed is:

1. A microfluidic detection device which has a spiral flow path and in which microfluid flows by a capillary phenomenon, the microfluidic detection device comprising:
an upper layer configured to receive a fluid sample through a first inlet portion that protrudes on an upper side of a first substrate, the upper layer having a plurality of flow paths which is provided in a lower side of the first substrate and in which a washing solution moves; and
a lower layer provided with a concave hole in an upper side of a second substrate such that the fluid sample is moved therethrough, the lower layer being configured to receive the fluid sample from a second inlet portion connected to the first inlet portion.

2. The microfluidic detection device of claim 1, wherein the upper layer comprises:
a pouch portion configured to supply the washing solution by a pressure;
a collecting portion in which a waste solution containing the washing solution that is used is collected;
a first spiral portion through which the waste solution passes;
a valve portion configured to adjust a speed of the washing solution;
a discarding portion in which the waste solution is stored; and
a vent portion provided on a first side of the discarding portion and configured to discharge the pressure.

3. The microfluidic detection device of claim 2, wherein the pouch portion comprises a washing fluid flow path in which the washing solution moves.

4. The microfluidic detection device of claim 2, wherein the valve portion is provided with a plurality of protrusions, and is coated with a hydrophobic material, thereby preventing backflow of the waste solution.

5. The microfluidic detection device of claim 2, wherein the vent portion is provided as a hole that penetrates the upper side of the first substrate.

6. The microfluidic detection device of claim 2, wherein the lower layer comprises:
a loading portion in which a detection antibody is loaded so that the detection antibody is mixed in the fluid sample;
a channel portion provided in a zigzag shape, the channel portion being configured to form a first complex by mixing the fluid sample and the detection antibody to each other;
a second spiral portion that is a flow path provided such that the first complex and the washing solution are sequentially moved; and
a coupling portion in which a capture antibody is loaded, the coupling portion having a predetermined depth such that a second complex is formed by coupling the first complex and the capture antibody to each other.

7. The microfluidic detection device of claim 6, further comprising:
a first flow path portion connecting the second inlet portion and the loading portion to each other; and
a second flow path portion connecting the loading portion and the channel portion to each other.

8. The microfluidic detection device of claim 6, wherein the coupling portion is configured such that the first complex and the capture antibody are filled in the coupling portion for a predetermined time and are coupled to each other.

9. The microfluidic detection device of claim 8, wherein the coupling portion is provided at a lower side of the collecting portion so that the fluid sample containing the detection body not coupled as the second complex is collected in the collecting portion.

10. The microfluidic detection device of claim 9, wherein the waste solution comprises:
the fluid sample containing the detection antibody not coupled as the second complex; and
the washing solution which is used and which contains impurities and bubbles.

11. The microfluidic detection device of claim 6, wherein the second spiral portion is configured to move the first complex and the washing solution to the coupling portion.

12. The microfluidic detection device of claim 11, wherein the second spiral portion connects the pouch portion and the coupling portion to each other, and is configured such that a height of a flow path of the second spiral portion is reduced from the coupling portion to the pouch portion, thereby forming a structure preventing backflow of the first complex and the washing solution.

13. The microfluidic detection device of claim 9, wherein the second spiral portion and the first spiral portion are provided such that a rotation direction of the second spiral portion and a rotation direction of the first spiral portion are opposite from each other.

14. The microfluidic detection device of claim 1, wherein the upper layer and the lower layer are coupled to each other by chemical coupling at room temperature.
